(19) United States
(12) Patent Application Publication
(10) Pub. No.: US 2024/0342092 A1
(43) Pub. Date: Oct. 17, 2024

(54) AMORPHOUS CALCIUM PHOSPHATE CERAMIC-BASED DRUG DELIVERY SYSTEM AND METHOD FOR ITS FABRICATION

(71) Applicant: RIGAS TEHNISKA UNIVERSITATE, Riga (LV)

(72) Inventors: Kristaps RUBENIS, Riga (LV); Dagnija LOCA, Riga (LV); Janis LOCS, Riga (LV); Signe SPERGA, Riga (LV)

(21) Appl. No.: 18/682,919

(22) PCT Filed: Jul. 26, 2022
(86) PCT No.: PCT/IB2022/056873

§ 371 Date: Feb. 11, 2024

(30) Foreign Application Priority Data
Aug. 10, 2021 (LV) LVP2021000045

(51) Int. Cl. A61K 9/16; A61K 9/14; A61K 31/7036; C04B 35/447; C04B 35/645

(52) U.S. Cl. CPC A61K 9/1694; A61K 9/143; A61K 31/7036; C04B 35/447; C04B 35/645

(57) ABSTRACT

The present innovation relates to the field of biomaterials, drug delivery systems, load-bearing materials for bone regeneration and dental restauration. The method for fabrication of amorphous calcium phosphate ceramic-based drug delivery system is proposed. According to the method, mixture of amorphous calcium phosphate and at least one drug is sintered at pressure that exceeds 500 MPa, preferably in the range of 600 to 1500 MPa, at temperature from 15 to 35° C. The selected pressure range allows to obtain on amorphous calcium phosphate ceramic-based long-term drug delivery system with excellent mechanical properties.

The present innovation relates to the field of biomaterials, specifically, to amorphous calcium phosphate ceramic-based drug delivery systems and methods for its fabrication. The innovation can be used in the following areas: drug delivery systems, load-bearing materials for bone regeneration and dental restauration.

### BACKGROUND ART

Amorphous calcium phosphate has excellent biocompatibility and better resorbability than hydroxyapatite—one of the most used bone substitute materials. Despite these characteristics, currently, the use of amorphous calcium phosphate in the field of biomaterials is limited—it is used only in the form of powder or coating. Due to its metastability, it is difficult to obtain amorphous calcium phosphate in a form of ceramics by sintering amorphous calcium phosphate powder [1]. The form of dense ceramic would significantly broaden application areas for the amorphous calcium phosphate.

Exceptional mechanical properties, chemical inertness and, often, excellent biocompatibility of ceramic materials makes them attractive materials for drug delivery systems. However, the potential to use ceramic materials in drug delivery systems is limited that is mainly related to temperatures that are necessary for their fabrication. The temperatures that are necessary for fabrication (sintering) of ceramic materials often exceeds 1000° C. while the drugs that are used in drug delivery systems usually cannot be exposed to temperatures that significantly exceeds room temperature (23° C.) for extended periods of time. Therefore, ceramic materials-based drug delivery systems are usually obtained by attaching or incorporating drugs into a previously fabricated ceramic material [2]. If fabrication of ceramic materials-based drug delivery systems would be possible in a single step by simultaneously sintering ceramic and drugs, it would be possible to obtain novel, long-term drug delivery systems with excellent mechanical properties.

By using the traditional ceramic materials sintering technique-heating the material being sintered to ˜80% of its melting temperature under ambient atmosphere, amorphous calcium phosphate cannot be sintered because at around 600-800° C. temperature it transforms to other calcium phosphate phases [3].

A method for sintering of amorphous calcium phosphate by using the so-called cold sintering process is known. To sinter powdered material, it is first mixed with a small amount of solvent. Afterward, the material is uniaxially compacted usually at temperatures from room temperature to ˜300° C. [4]. By using this method, amorphous calcium phosphate with 20 wt. % water added has been sintered to approximately 75% of its true density value (sintering was done at room temperature under uniaxial pressure of 500 MPa). At higher sintering temperatures (≥100° C.) it transformed to other calcium phosphate phases [5].

A method for sintering of amorphous calcium phosphate by uniaxial cold pressing (room temperature) and uniaxial hot pressing is known. By using this method, amorphous calcium phosphate has been sintered to approximately 75% of its true density value already at room temperature (sintering was done at 500 MPa). Relative density of the amorphous calcium phosphate ceramic obtained by hot uniaxial pressing (100 un 120° C., 500 MPa) was comparable to relative density of the ceramic obtained at room temperature [5].

A method for fabrication of amorphous calcium phosphate-based drug delivery system is known, where amorphous calcium phosphate powder is drug loaded by immersion in a phosphate buffer solution which contains drugs (doxorubicin). Doxorubicin from the phosphate buffer solution adsorbs on the surface of amorphous calcium phosphate, thus allowing to obtain drug delivery system in a form of powder. Such drug delivery system in a phosphate buffer solution releases 50% of its drug content within 72 h [6].

A method for fabrication of amorphous calcium phosphate-based drug delivery system is known, where amorphous calcium phosphate powder is soaked with alendronate (5.6 wt. %). Such drug delivery system in a N-(2-Hydroxyethyl) piperazine-N′-(2-ethanesulfonic acid) buffer released ˜18% of its drug content within 20 days [7].

Above-mentioned method by which amorphous calcium phosphate is sintered by uniaxial cold pressing is chosen as a prototype for the invention-amorphous calcium phosphate ceramic-based drug delivery system.

### THE OBJECT OF THE INVENTION

The aim of the present invention is to create a method for fabrication of amorphous calcium phosphate ceramic-based drug delivery system. Fabrication of such drug delivery system is possible at room temperature, thereby ensuring stability for amorphous calcium phosphate phase and preventing thermal degradation of the drugs. Such drug delivery system has high mechanical strength (comparable to calcium phosphate ceramics that are sintered at temperatures >1000° C.) and it can provide long-term drug delivery.

To achieve the aim of the invention, mixture of amorphous calcium phosphate and at least one drug is sintered at pressure that exceeds 500 MPa, preferably in the range of 600 to 1500 MPa, at temperature from 15 to 35° C. The selected pressure range allows to obtain on amorphous calcium phosphate ceramic-based long-term drug delivery system with excellent mechanical properties.

The term “amorphous calcium phosphate powder” generally refers to amorphous calcium phosphate powder with the following parameters: Ca/P ratio from 1.2 to 2, specific surface area 20-400 m²/g, true density 2.3-2.8 g/cm³, structural water content 0-20 wt. %.

The term “amorphous calcium phosphate ceramic” generally refers to amorphous calcium phosphate phase ceramic with a relative density (bulk density divided with true density) from 80 to 100%.

The term “sintering” generally refers to formation of homogeneous, mechanically durable structure when amorphous calcium phosphate particles bind together as a result of the applied pressure.

The term “room temperature” generally refers to temperature from 15 to 35° C.

The term “stabilized amorphous calcium phosphate” generally refers to amorphous calcium phosphate in whose structure contains different ions (carbonates, Mg, Sr, Zn etc.) that are incorporated to stabilize it.

The term “drug” generally refers to antibiotics, for example, gentamicin sulphate; osteoporosis medication, for example, strontium ranelate; growth factors, for example, bone morphogenetic protein; other medication that can be used in drug delivery systems.

### BRIEF DESCRIPTION OF THE DRAWINGS

FIG. 1. X-ray diffraction patterns: stability of amorphous calcium phosphate phase after its sintering at 500 to 1500 MPa pressure.

FIG. 2. Relative density of amorphous calcium phosphate ceramics as a function of pressure used for its sintering.

FIG. 3. Specific surface area of amorphous calcium phosphate ceramic as a function of pressure used for its sintering.

FIG. 4. Release of gentamicin sulphate from amorphous calcium phosphate ceramic obtained at 1500 MPa (gentamicin sulphate content 4 wt. %) over time in phosphate buffer solution.

FIG. 5. Release of strontium ranelate from amorphous calcium phosphate ceramics obtained at 1500 MPa (strontium ranelate content 17 wt. %) over time in phosphate buffer solution.

The method of the invention comprises the following sequential steps:

Amorphous calcium phosphate powder is mixed with at least one drug at a ratio of 1:0.01-0.2 by weight at room temperature. The amorphous calcium phosphate can be stabilized—it can contain different ions. Mixing of the amorphous calcium phosphate and drugs can be done manually by mortar and pestle or mechanically, for example, by ball mill for 1 to 5 min.

The mixture obtained is transferred to a pressing device by which pressure of 600 to 1500 MPa is applied to it. The pressure is increased at a rate of 10-100 MPa/min. The mixture is held under the pressure for 1 to 10 min. When the holding time ends, the pressure is slowly decreased and the obtained article which comprises of amorphous calcium phosphate and at least one drug is removed from the pressing device. Before the practical use, characterization of the obtained drug delivery system is done by methods known to the particular field.

### EXAMPLES

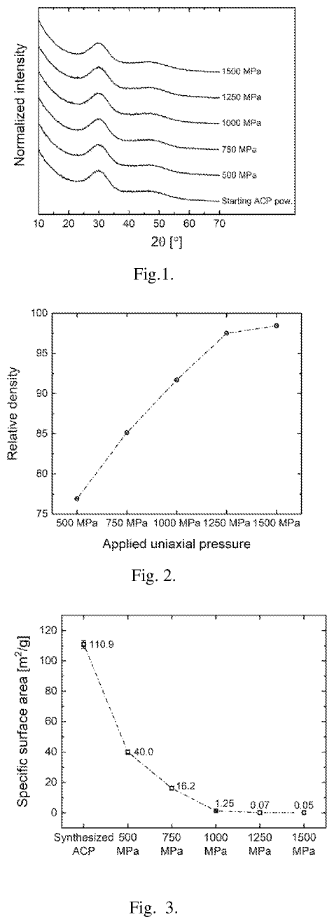

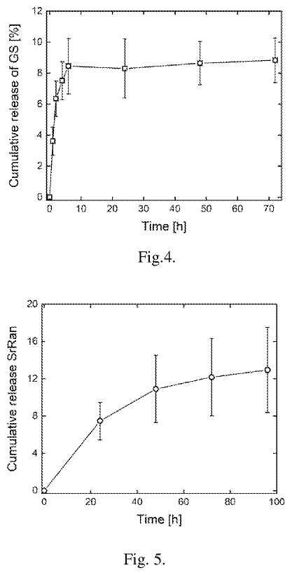

The following examples are intended to illustrate certain preferred embodiments of the invention and are not limiting in nature.
1) Amorphous calcium phosphate powder obtained by dissolution-precipitation method [8] was transferred to a pressing die and was subjected to uniaxial pressure of 500, 750, 1000, 1250 or 1500 MPa. The samples obtained were characterized by X-ray diffraction method and nitrogen sorptometry (used to determine specific surface area of the powder). In addition, their relative density was calculated (bulk density of the samples was divided with their true density value (2.52 g/cm³)) as well as their compression strength was determined. After compaction, all samples retained amorphous calcium phosphate phase (FIG. 1.). Relative density of the samples increased while specific surface area decreased with increase in pressure used for their compaction. Relative density of the samples compacted at 1500 MPa reached 98.44 (±0.02) % (FIG. 2.) while their specific surface area was more than 1000 times smaller than that of the starting powder (0.05 m²/g vs. 110.9 (±2.6) m²/g, respectively, FIG. 3.). Compression strength of the samples compacted at 1500 MPa reached 379 (±27) MPa.
2) Amorphous calcium phosphate powder (0.5 g) was mixed with gentamicin sulphate at a ratio of 1:0.04 by weight. The mixture was transferred to a pressing die and subjected to uniaxial pressure of 1500 MPa. As a result, dense, gentamicin sulfate containing amorphous calcium phosphate ceramic was obtained. Gentamicin sulphate release experiments were done in phosphate buffer solution. Approximately 9% of the gentamicin content was released from the ceramic samples within 72 h (FIG. 4.).
3) Amorphous calcium phosphate powder (0.5 g) was mixed with strontium ranelate at a ratio of 1:0.2 by weight. The mixture was transferred to a pressing die and subjected to uniaxial pressure of 1500 MPa. As a result, dense, strontium ranelate containing amorphous calcium phosphate ceramic was obtained. Strontium ranelate release experiments were done in phosphate buffer solution. Approximately 13% of the strontium ranelate content was released from the ceramic samples within 96 h (FIG. 4.).

### REFERENCES

[1] C. Combes and C. Rey, “Amorphous calcium phosphates: Synthesis, properties and uses in biomaterials,” Acta Biomater., vol. 6, no. 9, pp. 3362-3378 September 2010, doi: 10.1016/j.actbio.2010.02.017.
[2] M. Parent, H. Baradari, E. Champion, C. Damia, and M. Viana-Trecant, “Design of calcium phosphate ceramics for drug delivery applications in bone diseases: A review of the parameters affecting the loading and release of the therapeutic substance,” J. Control. Release, vol. 252, pp. 1-17, April 2017, doi: 10.1016/j.jconrel.2017.02.012.
[3] J. Vecstaudza, M. Gasik, and J. Locs, “Amorphous calcium phosphate materials: Formation, structure and thermal behaviour,” J. Eur. Ceram. Soc., vol. 39, no. 4, pp. 1642-1649 April 2019, doi: 10.1016/j.jeurceramsoc.2018.11.003.
[4] J. Guo et al., “Cold Sintering: A Paradigm Shift for Processing and Integration of Ceramics,” Angew. Chemie Int. Ed., vol. 55, no. 38, pp. 11457-11461, September 2016, doi: 10.1002/anie.201605443.
[5] K. Rubenis, S. Zemjane, J. Vecstaudza, J. Bitenieks, and J. Locs, “Densification of amorphous calcium phosphate using principles of the cold sintering process,” J. Eur. Ceram. Soc., vol. 41, no. 1, pp. 912-919, January 2021, doi: 10.1016/j.jeurceramsoc.2020.08.074.
[6] W. Feng et al., “An amorphous calcium phosphate for drug delivery: ATP provides a phosphorus source and microwave-assisted hydrothermal synthesis,” Mater. Today Commun., vol. 25, no. July, p. 101455, December 2020, doi: 10.1016/j.mtcomm.2020.101455.
[7] R. Sun et al., “Highly Porous Amorphous Calcium Phosphate for Drug Delivery and Bio-Medical Applications,” Nanomaterials, vol. 10, no. 1, p. 20, December 2019, doi: 10.3390/nano10010020.
[8] J. Vecstaudza and J. Locs, “Novel preparation route of stable amorphous calcium phosphate nanoparticles with high specific surface area,” J. Alloys Compd., vol. 700, pp. 215-222, 2017, doi: 10.1016/j.jallcom.2017.01.038.

1. The method for fabrication of amorphous calcium phosphate ceramic-based drug delivery system which comprises sintering of a mixture of amorphous calcium phosphate powder and at least one drug, characterized in that sintering occurs at temperatures from 15 to 35° C. under uniaxial pressure that exceeds 600 MPa.

2. The method according to claim 1, wherein amorphous calcium phosphate is stabilized.

3. The method according to claim 1, wherein the ratio of amorphous calcium phosphate to drugs are 1:0.01-0.2 by weight.

4. Amorphous calcium phosphate ceramic-based drug delivery system, that is obtained according to claim 1, comprises amorphous calcium phosphate and gentamicin sulphate.

5. Drug delivery system according to claim 4, for use in bone regeneration.

6. Drug delivery system according to claim 4, for use in dental restauration.
